(19) United States
(12) Patent Application Publication
(10) Pub. No.: US 2023/0172744 A1
(43) Pub. Date: Jun. 8, 2023

(54) AUTOMATIC EXCRETION-PROCESSING DEVICE, MANAGEMENT SYSTEM, DETERMINATION METHOD, AND PROGRAM

(71) Applicants: The University of Tokyo, Bunkyo-ku, Tokyo (JP); LIBERTYSOLUTION CO., LTD., Matsue-shi, Shimane (JP)

(72) Inventors: Masaki SEKINO, Tokyo (JP); Yasuhiro OKADA, Tokyo (JP); Yuki SUNAGA, Tokyo (JP); Tomio ADACHI, Sakaiminato-shi (JP)

(21) Appl. No.: 17/912,010

(22) PCT Filed: Mar. 10, 2021
(86) PCT No.: PCT/JP2021/009542

§ 371 Date: Sep. 15, 2022

Related U.S. Application Data
(60) Provisional application No. 62/991,082, filed on Mar. 18, 2020.

(51) Int. Cl. A61F 5/44; A61B 10/00; G06T 7/90; G06V 10/44; G06V 10/74; G06V 10/764

(52) U.S. Cl. CPC A61F 5/44; A61B 10/007; A61B 10/0038; G06T 7/90; G06V 10/44; G06V 10/761; G06V 10/764; G06T 2207/10024; G06T 2207/20084

(57) ABSTRACT

An automatic excretion-processing device (1) includes a cup (2) attached to a human body and configured to receive an object which is excreted from the human body, a processing unit (3) configured to transfer the object in the cup outside of the cup, an acquisition unit (4) configured to image the inside of the cup and to acquire a captured image of the object, and a determination unit (5) configured to extract a feature of the object with reference to a result of iterative learning using the captured images of the object in different states on the basis of the captured images acquired by the acquisition unit and to classify the state of the object.

### TECHNICAL FIELD

The present invention relates to an automatic excretion-processing device, a management system, a determination method, and a program.

Priority is claimed on U.S. Pat. Application No. 62/991,082, filed Mar. 18, 2020, the content of which is incorporated herein by reference.

### BACKGROUND ART

Recently, the number of care-requiring persons such as aged persons, physically handicapped persons, or patients who need assistance for excretion processing has been increasing every year. The number of care-requiring persons is increasing, but the number of care workers is chronically insufficient. This trend is predicted to become more significant in the future. Excretion processing of care-requiring persons imposes a large burden on care workers. An automatic excretion-processing device that assists with excretion of care-requiring persons to reduce the burden on care workers has been proposed.

A state of an object excreted from a care-requiring person is inspected by a person engaged in care to manage conditions of the care-requiring person. For example, a technique described in Patent Literature 1 is known to manage an object excreted from a human body. The technique described in Patent Literature 1 includes an imaging unit that acquires a captured image of overflowed water in a drain pipe of a toilet bowl and a calculation unit that calculates a volume of a liquid object excreted from a human body on the basis of the captured image.

### CITATION LIST

### Patent Literature

[Patent Literature 1] Japanese Unexamined Patent Application, First Publication No. 2018-109285

### Non-Patent Literature

[Non-Patent Literature 1] E. Halmos, J. Biesiekierski, E. Newnham, and P. R. Gibson: “Inaccuracy of patient-reported descriptions of and satisfaction with bowel actions in IBS,” Journal of Nutrition &amp; Intermediary Metabolism (2017)
[Non-Patent Literature 2] S. J. Lewis, K. W. Heaton: “Stool Form Scale as a Useful Guide to Intestinal Transit Time”, Scand. J. Gastroenterol. 32,920-924 (1997)
[Non-Patent Literature 3] Y. Le Cun, B. Boser, J. S. Denker, D. Henderson, R. E. Howard, Hubbard, W., Jackel, L. D.: “Backpropagation applied to handwritten zip code recognition,” Neural computation, 1(4), pp. 541-551, (1989)
[Non-Patent Literature 4] Ramprasaath R Selvaraju, Michael Cogswell, Abhishek Das, Ramakrishna Vedantam, Devi Parikh, Dhruv Batra: “Grad-CAM: Visual Explanations from Deep Networks via Gradient-based Localization,” The IEEE International Conference on Computer Vision (ICCV), pp. 618-626 (2017)

### SUMMARY OF INVENTION

### Technical Problem

The technique described in Patent Literature 1 is for estimation of a volume of a liquid object, but is not for determining a state of a solid object. The inventor et al. have earnestly studied an automatic excretion-processing device that automatically determines a state of a solid object to reduce a burden on care workers.

### Solution to Problem

According to an aspect of the present invention, an automatic excretion-processing device is provided, including: a cup attached to a human body and configured to receive an object which is excreted from the human body; a processing unit configured to transfer the object in the cup outside of the cup; an acquisition unit configured to image the inside of the cup and to acquire a captured image of the object; and a determination unit configured to extract a feature of the object with reference to a result of iterative learning using the captured images of the object in different states on the basis of the captured images acquired by the acquisition unit and to classify the state of the object.

According to the present invention, since the acquisition unit acquires the captured images of the object excreted into the cup, it is not necessary for a person engaged in care to inspect the state of the object and it is possible to reduce a burden on the person engaged in care and to improve a labor environment. The acquired captured images are analyzed to determine the state of the object by the determination unit. The determination unit has been subjected to iterative machine learning to determine the state of the object in advance. Accordingly, the determination unit can recognize the object in the captured images. The determination unit can recognize the features of the object in the captured images. The determination unit is configured to recognize the features of the object through learning corresponding to patterns classified in advance. When the features of the object are recognized, the determination unit classifies the state of the object according to the features. The person engaged in care can use the classification result from the determination unit to manage conditions of a care-requiring person.

In the present invention, the determination unit may be configured to monitor temporal change of the object in the cup on the basis of the captured images and to classify the state of the object.

Since it may be difficult to extract the feature of the object using only one captured image, the determination unit can ascertain a change of the object by monitoring the temporal change of the object in the cup.

In the present invention, the determination unit may be configured to classify the state of the object on the basis of a degree of deformation of the object when the object reaches the inside of the cup and is being deformed on the basis of the captured images.

A solid object has a viscosity varying according to a water content thereof. When the object is excreted from a human body and reaches a wall of the cup, the object is deformed. Since a degree of deformation with time varies according to the viscosity of the object, the determination unit can classify the object according to the degree of deformation with reference to a result of learning of the degree of deformation with time of the object.

In the present invention, the determination unit may be configured to classify a symptom of the human body by comparing a color of the object with a reference on the basis of the captured images.

In addition to the classification based on the viscosity of the object, a color of the object can be used as a determination matter for ascertaining a change in conditions of a care-requiring person. The determination unit can determine, for example, a feature in which blood is included in the object on the basis of a color of a captured image. Since the color of blood varies with time, a bleeding part can be estimated according to a color depth of the blood and a symptom of a care-requiring person can be classified. The determination unit can determine a feature in which an undigested object is included in the object on the basis of the color of the captured image.

In the present invention, the determination unit may be configured to classify a symptom of the human body by comparing a color of the object with a reference on the basis of the captured images and to determine a coping method corresponding to the symptom.

According to the present invention, when a symptom of a care-requiring person is classified on the basis of the color of the object, a person engaged in care can carry out medication or treatment on the care-requiring person promptly by causing the determination unit to determine a coping method corresponding to the symptom.

In the present invention, the determination unit may be configured to extract the feature of the object through the iterative learning in which deep learning using a convolutional neural network is performed and to classify the state of the object.

The determination unit can extract the feature of the object on the basis of the captured images by performing deep learning using a convolutional neural network able to handle image classification in machine learning.

In the present invention, the determination unit may be configured to classify a symptom of the human body by comparing the number of appearances of the object in a predetermined period with a reference.

When the frequency of excretion of the care-requiring person decreases, symptoms such as constipation and intestinal obstruction are suspected. It is possible to manage conditions of the care-requiring person by causing the determination unit to ascertain the frequency of excretions of the care-requiring person.

According to another aspect of the present invention, a management system is provided, including: an automatic excretion-processing device including a cup attached to a human body and configured to receive an object which is excreted from the human body, a processing unit configured to transfer the object in the cup outside of the cup, and an acquisition unit configured to image the inside of the cup and to acquire a captured image of the object; and a management device configured to acquire a captured image from the automatic excretion-processing device via a network, to extract a feature of the object with reference to a result of iterative learning using the captured images of the object in different states on the basis of the captured images acquired by the acquisition unit and to classify the state of the object.

When machine learning is performed on the basis of captured images of an object, it is difficult to collect the captured images serving as training data. With the management system according to the present invention, a plurality of automatic excretion-processing devices that acquire a captured image can be connected to a network and acquire a large number of captured images required for learning. Since many captured images can be acquired with extension of an operation period of the management system, the determination unit can perform supervised learning. The determination unit may start its operation with unsupervised learning and increase opportunities of learning with extension of the operation period of the management system to improve determination accuracy.

According to another aspect of the present invention, a determination method that is performed by a computer is provided, the determination method including: imaging the inside of a cup in an automatic excretion-processing device including a cup attached to a human body and configured to receive an object which is excreted from the human body and a processing unit configured to transfer the object in the cup outside of the cup; acquiring a captured image of the object; and extracting a feature of the object with reference to a result of iterative learning using the captured images of the object in different states on the basis of the acquired captured images and classifying the state of the object.

With the determination method according to the present invention, the determination unit can recognize a feature of the object through learning corresponding to patterns classified in advance and classify the state of the object according to the feature.

According to another aspect of the present invention, a program is provided causing a computer to perform: imaging the inside of a cup in an automatic excretion-processing device including a cup attached to a human body and configured to receive an object which is excreted from the human body and a processing unit configured to transfer the object in the cup outside of the cup; acquiring a captured image of the object; and extracting a feature of the object with reference to a result of iterative learning using the captured images of the object in different states on the basis of the acquired captured images and classifying the state of the object.

With the program according to the present invention, it is possible to recognize the feature of the object through learning corresponding to patterns classified in advance and to classify the state of the object according to the feature.

### Advantageous Effects of Invention

According to the present invention, it is possible to provide an automatic excretion-processing device, a management system, a determination method, and a program that can determine a state of an object by extracting features of a captured image of the object.

### BRIEF DESCRIPTION OF DRAWINGS

FIG. 1 is a block diagram illustrating an example of a configuration of an automatic excretion-processing device.

FIG. 2 is a diagram illustrating an example of the configuration of the automatic excretion-processing device.

FIG. 3 is a diagram illustrating an example of a method of classifying a type of a measurement object.

FIG. 4 is a diagram illustrating an example of a result of classification of a measurement object based on captured images.

FIG. 5 is a diagram illustrating an example of a routine which is performed in a determination method.

FIG. 6 is a diagram illustrating an example of a routine which is performed in another determination method.

FIG. 7 is a diagram illustrating a correct answer rate of test data based on the determination methods.

FIG. 8 is a diagram illustrating a determination process in another determination method.

FIG. 9 is a diagram illustrating a determination process in a determination method.

FIG. 10 is a diagram illustrating a correct answer rate of determination results when prior learning is applied to the determination methods.

FIG. 11 is a flowchart illustrating a routine which is performed by the automatic excretion-processing device.

FIG. 12 is a diagram illustrating an example of a configuration of a management system.

### DESCRIPTION OF EMBODIMENTS

### First Embodiment

Hereinafter, an automatic excretion-processing device, a management system, a determination method, and a program according to an embodiment of the present invention will be described. The automatic excretion-processing device according to the present invention is a device that automatically determines mainly a state of a solid object excreted from a human body of a care-requiring person when excretion processing is automatically performed.

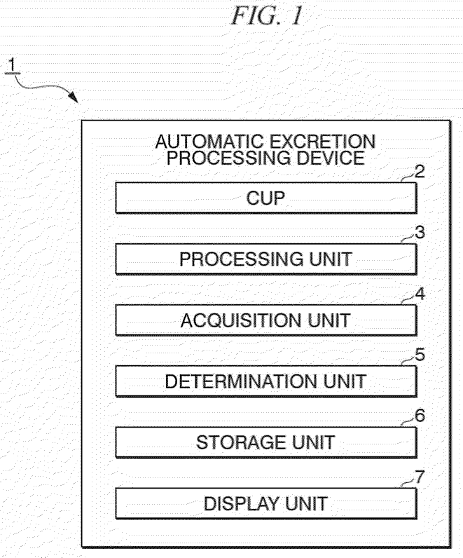

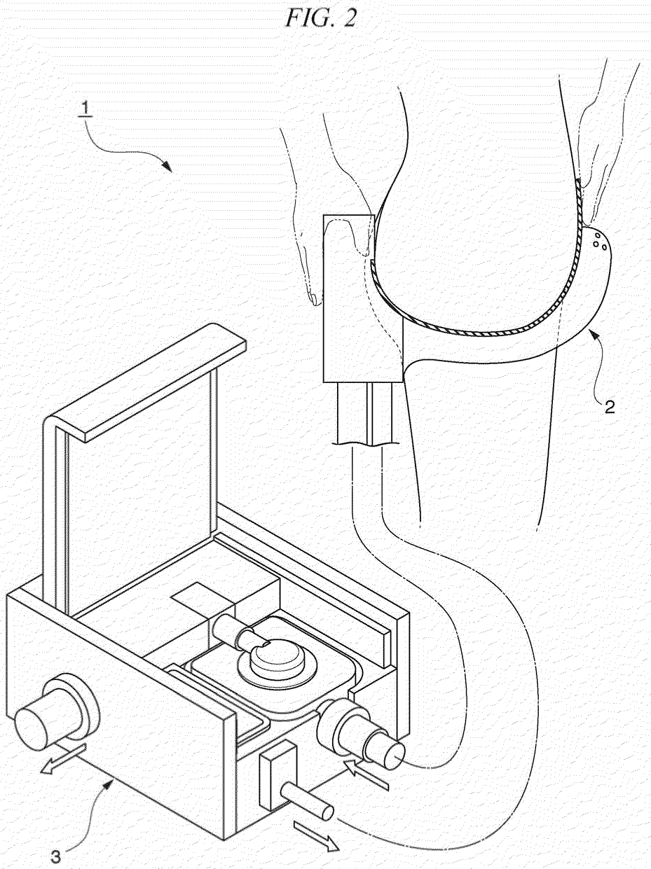

As illustrated in FIGS. 1 and 2, an automatic excretion-processing device 1 includes a cup 2 that is attached to a human body, a processing unit 3 that transfers an object in the cup outside of the cup 2, an acquisition unit 4 that images the inside of the cup and acquires the captured image of the object, a determination unit 5 that determines a state of the object on the basis of the captured image, a storage unit 6 that stores various types of data, and a display unit 7 that displays a determination result.

The cup 2 is formed to receive an object excreted from a human body of a care-requiring person. The processing unit 3 performs a process of transferring the object received in the cup 2 outside of the cup and washing the inside of the cup 2 and the human body of the care-requiring person. The processing unit 3 supplies washing water and air to the inside of the cup 2. The processing unit 3 transfers the object, the washing water, and the air from the inside of the cup 2 outside of the cup 2 through suction. The processing unit 3 temporarily stores the object transferred from the inside of the cup 2. The processing unit 3 may discharge the object to a drain pipe such as a sewer pipe. The acquisition unit 4 includes a camera that images the inside of the cup 2.

The camera is attached to, for example, the cup 2 to image the inside of the cup 2 and consecutively acquires electronic captured images. The camera is, for example, an endoscope camera including a light source. The acquisition unit 4 may include an infrared camera without including a light source instead of an endoscope camera. The acquisition unit 4 may further include another sensor such as an ultrasonic sensor that generates an ultrasonic image. The acquisition unit 4 stores the captured images in the storage unit 6.

The determination unit 5 reads the captured images acquired by the acquisition unit 4 and stored in the storage unit 6. The determination unit 5 has been subjected to iterative learning using different captured images of objects in advance and extracts features of an object with reference to a learning result on the basis of the captured image acquired by the acquisition unit 4.

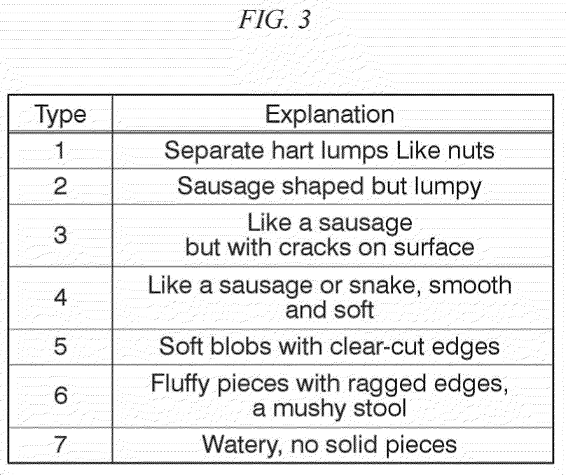

As illustrated in FIG. 3, the determination unit 5 classifies a state of the object on the basis of the extracted features. The classification is the Bristol stool scale (hereinafter appropriately referred to as BSFS) used in the field of medicine (see Non-Patent Literature 1). As illustrated in the drawing, the Bristol stool scale is a medical diagnostic tool that classifies a state of an object into seven categories.

The determination unit 5 is realized, for example, by causing a processor such as a central processing unit (CPU) to execute a program stored in a program memory. Some or all of the determination unit 5 may be realized by hardware such as a large-scale integration (LSI), an application-specific integrated circuit (ASIC), or a field-programmable gate array (FPGA) or may be realized by combination of software and hardware.

The storage unit 6 is realized, for example, by a hard disc drive (HDD), a flash memory, an electrically erasable programmable read-only memory (EEPROM), a read-only memory (ROM), or a random-access memory (RAM) or a hybrid storage device using two or more thereof. Various programs such as firmware and application programs, processing results from various functional units, and the like are stored in the storage unit 14.

The display unit 7 is an image display device using a liquid crystal display, an organic EL display, or the like. The determination unit 5, the storage unit 6, and the display unit 7 may be constituted by a personal computer, a tablet terminal, or a smartphone which is separate from the automatic excretion-processing device.

Processing of the determination unit 5 will be described below.

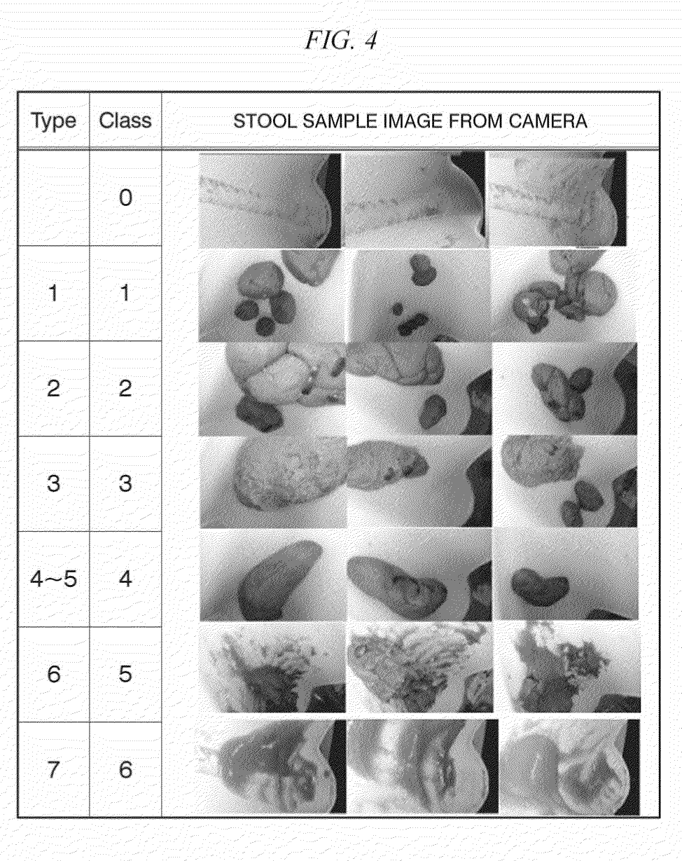

As illustrated in FIG. 4, training data for an object was prepared to verify the determination accuracy of the determination unit 5. A pseudo sample for reproducing various states and shapes using soybean paste, soft wheat flour, or chocolate powder as the object was prepared. Training data sets (700 sets) and test data sets (171 sets) were prepared to classify a shape of an object. In this test, according to the BSFS, shape regularity for each type was determined, the sample was finely tuned using soybean and soft wheat flour, and texture was reproduced.

Since the states of the objects were not easily distinguished between Type 4 and Type 5 in the BSFS, the states of the objects were integrated into Class 4, a state in which nothing appears was defined as Class 0, and the states of the objects were reclassified into a total of seven classes. Since proportions of soybean and soft wheat flour of samples for reproducing Types 1 to 4 are tuned to be constant, water contents thereof are constant. The reason the water contents are made to be constant is that there is a correlation between a shape of an object and water content of the object as an index of the BSFS (for example, see Non-Patent Literature 2). Accordingly, it is thought that the water contents of the samples of the objects can be estimated by classifying the objects on the basis of shapes thereof. Then, a state in which an object had been excreted inside of the cup 2 was reproduced, and imaging for collecting image data was performed. In the image data, temporal changes of the states of the samples in the cup 2 when the prepared samples fell from a position corresponding to an anus part were imaged.

The determination unit 5 monitors a change of a solid object with time in the cup on the basis of the captured images and classifies the state of the object on the basis of Bristol stool scale. In this classification, the captured images are analyzed, and elements which need to be analyzed for estimating health conditions and a reference are provided. In this embodiment, images of the object are collected by the camera provided in the cup. In determination which is performed by the determination unit 5, analysis is performed on the basis of the following elements which are normally ascertained in a care facility in which the automatic excretion-processing device is used.
(1) A shape classification of an object and a change thereof
(2) A color of the object or blood or mixture included therein
(3) An excretion frequency or a volume of the object

The determination unit 5 extracts a feature of the object, for example, on the basis of iterative learning in which deep learning using a convolutional neural network (CNN) (see Non-Patent Literature 3) is performed and classifies the state of the object. The CNN which is performed by the determination unit 5 is, for example, learning using the captured images of the object classified and prepared in advance as training data. In this embodiment, two types of CNN models were compared to verify the determination accuracy.

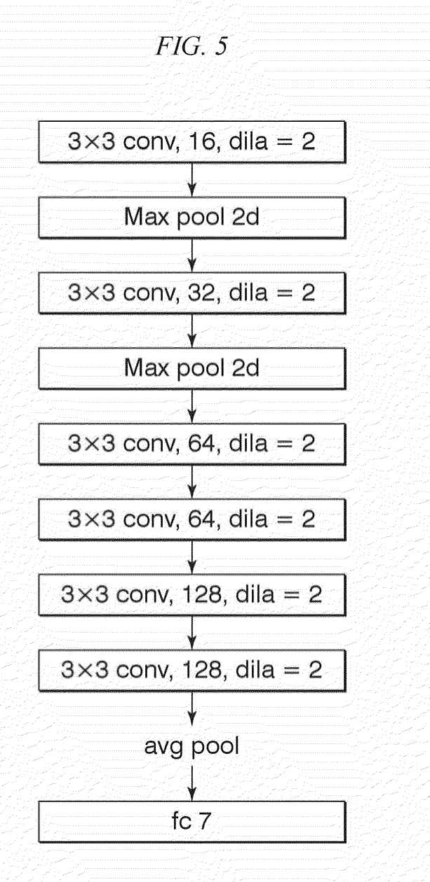

FIG. 5 schematically illustrates a routine which is performed in a Simple model which is a first CNN model. The Simple model is, for example, a model with a basic configuration for performing a process of extracting a shape on the basis of the captured images. The Simple model includes six convolutional layers for performing a process of extracting a shape from a captured image and an avg pooling layer for compressing information of input data after processing in the convolutional layers.

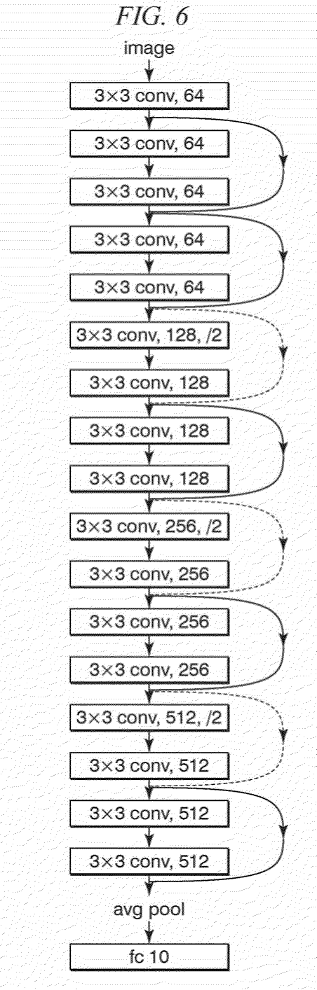

FIG. 6 schematically illustrates a routine which is performed in a transfer learning model (Resnet18) which is a second CNN model (for example, see Non-Patent Literature 3). Resnet18 is a general transfer learning model. The transfer learning model is an image classification network subjected to prior learning. The network of the transfer learning model is subjected to prior learning such that a feature is extracted from an image. In prior learning, learning for extracting a feature which is strong and which has a large amount of information is performed in advance on the basis of a large number of natural images. The Resnet18 is configured to process a convolutional neural network of 18 layers subjected to prior learning for extracting a feature on the basis of the captured images using a large number of data sets.

When parameters in the transfer learning model are finely tuned, the transfer learning model can be applied to various data sets or applications. In this test, all the parameters were changed using the Resnet18 (full-fine tuning).

Since the Simple model has shallower layers and poorer power of expression than the Resnet18 but has fewer parameters, the Simple model can have a calculation cost reduced and be applied to a 3D CNN. In the following test, cross entropy was used as a classification loss and a stochastic gradient descent method with a damping learning rate of 0.001 was used as a gradient method.

In a pre-process of image recognition, data extension for increasing the number of pieces of image data used as training data is performed. The data extension is a general technique of increasing the unevenness of training data and decreasing an algorithm bias of data sets. The data extension is applied in a stage in which training data is input. In the data extension, processes such as rotation, reversal, trimming, and zooming of image data are generally performed, and the original number of pieces of data can be increased. For the purpose of facilitating learning based on input images, two pre-processes including gamma correction and histogram equalization were performed on the input images.

The gamma correction is correcting grayscales of an image according to an appropriate curve of gamma values such that a final output I′(x, y) conforms to Expression (1), where Iₘₐₓ, γ defines a maximum value of pixel values and I(x, y) defines pixel values of an input image. In this test, since the inside of the cup 2 which has been imaged is shaded, the gamma correction was performed with γ=0.5.

$$\text{y} \text{=} \text{255} ∗ {\left(\frac{x}{255}\right)}^{\frac{1}{y}}$$

In the histogram equalization, histograms of pixel values of the whole input image are equalized. Thereafter, the frequencies of all the pixels (0 to 255) are converted to the average number of pixels. Through this process, it is possible to improve the contrast of the image and to adjust a light source environment. Specifically, when pixel values of an input image are defined as I(x, y), the frequencies thereof are defined as H(x, y), and the total number of pixels of the input image is defined as S, the pixel values I′(x, y) of an output image can be calculated by Expression (2).

$${I}^{′} \left(x , y\right) = \frac{{I}_{m a x}}{s} {\sum }_{i = 0}^{I \left(x , y\right)} H \left(x , y\right)$$

In this test, image data was divided into subareas of 8×8 pixels and the histograms for each subarea were averaged. The grounds for determination for deriving the classification result may not be visible in the CNN according to the related art. In the CNN, rich features have been ascertained in the processes in a plurality of convolutional layers. For example, when gradient-weighted class activation mapping (Grad-CAM) described in Non-Patent Literature 4 is used, it is possible to visualize what part of a feature map has contributed to the classification by calculating gradients of the convolutional layers. The Grad-CAM calculates weights of feature images on the basis of Expression (3) using the gradient at the time of back propagation.

$${α}_{k}^{c} = \frac{1}{Z} {\sum }_{i} {\sum }_{j} \frac{\partial {y}^{c}}{\partial {A}_{i j}^{k}}$$

In Expression (3),

$${α}^{\text{c}} \text{k}$$

is a partial differential of a probability y^{c} that an output feature image A^{k} of the CNN is determined to be class c. The gradient expressed by Expression (3) indicates an influence of a probability that class c will be determined on a pixel change at a position i and j in a feature map A^{k}ᵢⱼ. It is described that this gradient is the same as a weighted sum of feature images of the final layer (feature images based on class activation mapping (CAM)) (see Non-Patent Literature 4). Since a network of a model does not need to be changed unlike the CAM according to the related art, this method can be applied to various models.

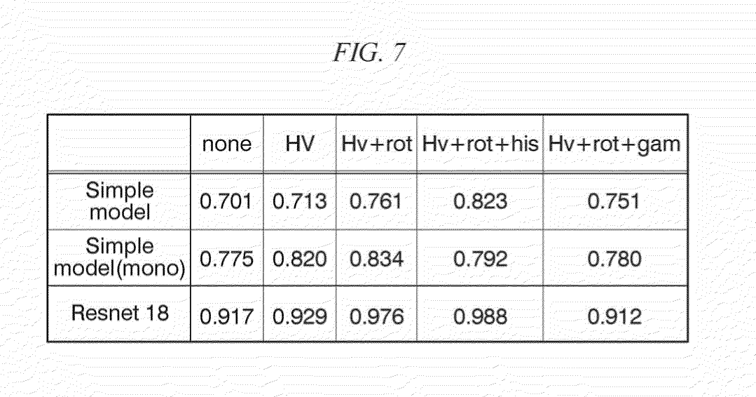

FIG. 7 illustrates a result of comparison between correct answer rates of test data based on the Resnet18 and the Simple model. The Resnet18 and the Simple model were trained under the conditions of pre-processing of training data and data extension thereof. In the drawing, HV and rot indicate image data which has been reversed or rotated (maximum 45 °), and his and gam indicate averaging and y conversion of histograms. Mono indicates an image processed in grayscales.

Regarding the correct answer rates of the Simple model and the Resnet18, the correct answer rate in transfer learning using the Resnet18 is higher by about 0.2 than that using the Simple model. This is because the Resnet18 includes a “discerning eye” provided by prior learning using ImageNet in addition to a depth of layers to be learned in advance and thus has a higher performance than the Simple model.

In the Simple model, the result of determination based on images in grayscale is higher in performance when data extension has not been performed in comparison with the result of determination based on images in the RGB color space. In the Simple model, performance when histograms of images were averaged is higher than when the images were handled in the RGB color space. When captured images were vertically reversed and rotated and training data was extended, accuracy in any model was improved. The correct answer rate when histograms were averaged is higher in an increase width than when gamma correction of the images was performed. The correct answer rate of the Simple model is 82.3%, which is high accuracy. The correct answer rate of the transfer learning using the Resnet18 is 98.8, which is much higher accuracy than the Simple model.

Then, feature images serving as the bases for determination for each output class of the Simple model and the Resnet18 model are extracted. The Simple model and the Resnet18 model are trained on the basis of training data subjected to data extension and output feature images serving as the bases of determination using the Grad-CAM.

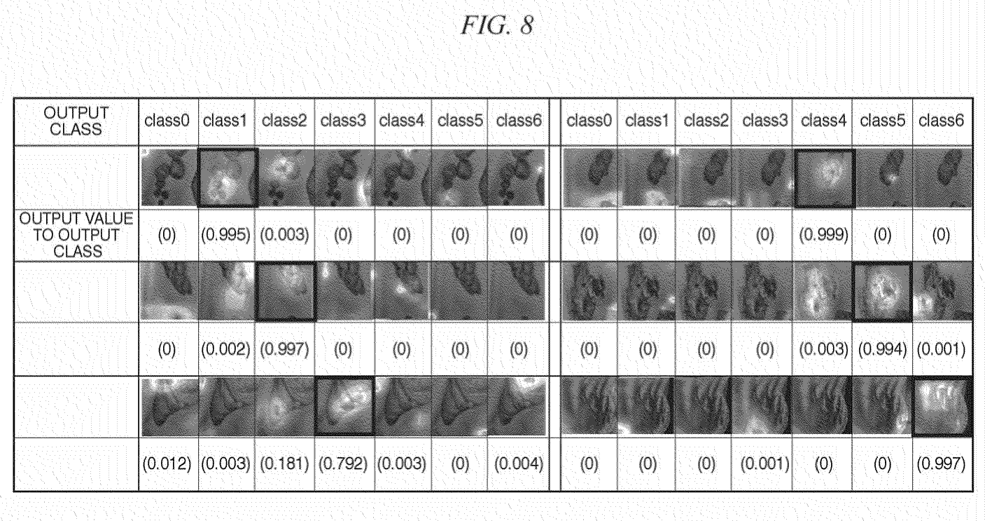

FIG. 8 illustrates bases of determination for sample images of classes 1 to 6 based on the Resnet18 model. As illustrated in the drawing, the order (gradient) of the output classes of the feature images surrounded by black frames matches the correct answer label. In the feature images, a part deep in color is considered as being important, and output values under the images thereof are output to the output classes. For example, for an image of which a correct answer is class 1 in the feature images of “output class,” the determination unit 5 determines that all the images of the object are the bases for determination, and outputs an output value (0.995). Similarly, for images of which a correct answer ranges from class 2 to class 6, the determination unit 5 determines that all the images of the object are the bases for determination, and outputs a maximum value to the correct answer label of the “output class.” From the aforementioned description, the Resnet18 model can classify the captured images of the object according to the order of classification of the BSFS in the process of determining a feature value in an image of the object.

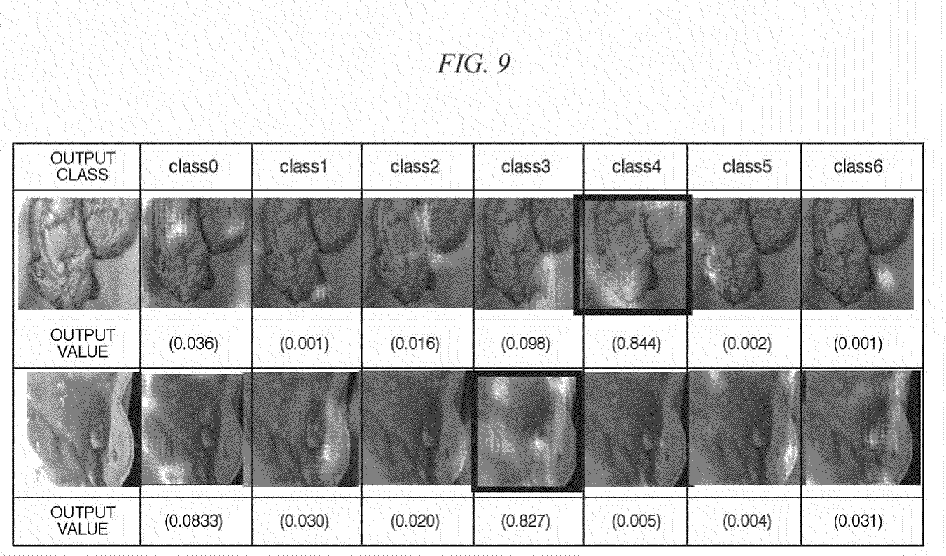

FIG. 9 illustrates bases for determination based on the Simple model. As illustrated in the drawing, the order (gradient) of the output feature images in the Simple model does not correspond to the order of classes and is nonlinear. It is necessary to extract feature values of images on the basis of an influence of surroundings of a feature part. Accordingly, the Simple model does not learn capability of “discerning images” in the process of determination and thus it can be seen that the gradient of feature images is nonlinear.

The Resnet18 has capability of extracting a feature value from an image through prior learning in advance, and the Simple model has lower learning capability of recognizing images with data sets of images of the object which are unevenly distributed than the Resnet18.

As described above, there is a large difference in accuracy between the Resnet18 model and the Simple model. Two reasons thereof are considered. One reason is that the layers of the Simple model are shallower than the Resnet18 model. The other reason is that the number of data sets used to extract feature values from images is small. Therefore, a model having the same structure as the Resnet18 and the Simple model are trained in advance using cifar-10 which is a sample image data set and are retrained using an image data set of the object, and the models are compared in performance.

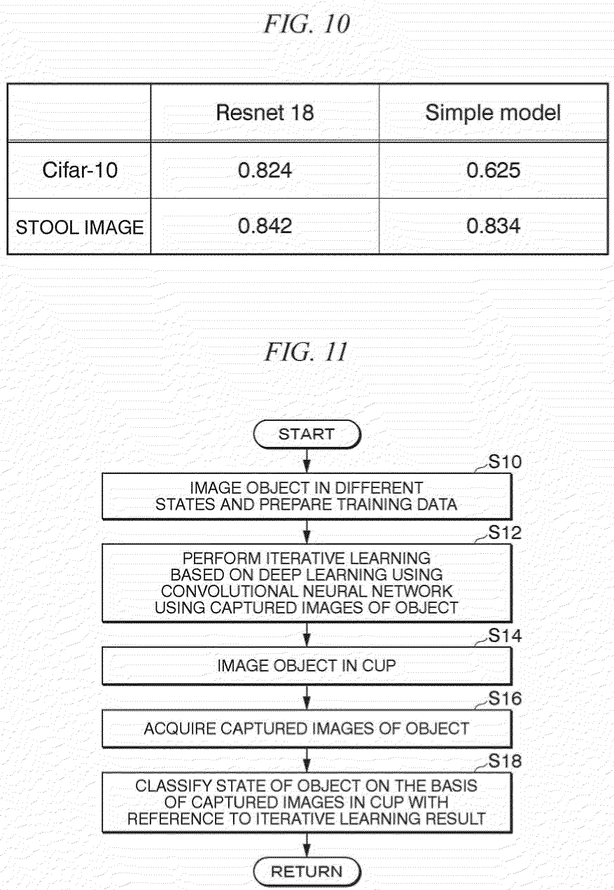

FIG. 10 illustrates correct answer rates of the determination result when prior learning is applied to the determination methods. As illustrated in the drawing, it was proved that performance of the Simple model is not improved in spite of prior learning. On the other hand, the Resnet18 model can classify images of the object into seven classes with accuracy of 98.8% through transfer learning. In the drawing, the pre-processing of image data of the cifar-10 includes lateral reversal and trimming, and the pre-processing of image data of the object includes vertical/lateral reversal, rotation, and histogram equalization.

However, when a state of an object in the cup 2 is determined on the basis of the Resnet18 model, the following problem is conceivable. In the automatic excretion-processing device 1, the cup 2 which is attached to a body of a care-requiring person has a small capacity. The automatic excretion-processing device 1 is configured to detect that an object falls in the cup 2 and to suction the object outside of the cup 2 at the same time.

Since a shape of a part of an object in the cup 2 is imaged, a water content needs to be estimated from the shape of a part of the object in order to determine the state of the object in the cup 2. Since the current BSFS is a technique of analyzing the state on the basis of the whole shape of the object, determination based on data in which a part of the object in the cup 2 is imaged may decrease in accuracy. Particularly, it is thought that distinction of type 1 and type 2 in the cup 2 is difficult. Therefore, a 3D model for causing the determination unit 5 to classify a state of an object by machine-learning a change with time of the object on the basis of moving image data at the time of excretion is proposed.

Since the determination unit 5 can also consider a viscosity based on a diffusion speed of the object by performing determination based on a moving image, it is possible to classify an object with high accuracy even when the determination is performed on the basis of data in which a part of the object is imaged. On the basis of the result of test, the Simple model or a CNN model subjected to transfer learning is applied to the determination unit 5. The CNN model subjected to transfer learning has higher accuracy than that of the Simple model. However, the CNN model subjected to transfer learning has an increased calculation time compared with the Simple model. Accordingly, when the Simple model is applied to the determination unit 5, it is preferable that the Simple model be extended to enhance classification accuracy.

The determination unit 5 perform classification on the basis of a change with time of an object recorded with captured images obtained by continuously imaging the inside of the cup 2 attached to a body of a care-requiring person using the Simple model or the CNN model subjected to transfer learning. The determination unit 5 determines the state of the object in the cup 2 on the basis of the change with time. The determination unit 5 extracts features of the object on the basis of the captured images acquired by the acquisition unit 4 with reference to a result of iterative learning using other captured images of the object in different states, and classifies the state of the object. The determination unit 5 monitors a change with time of the object in the cup on the basis of the captured images and classifies the state of the object.

The determination unit 5 monitors, for example, a state in which an object reaches an inner wall of the cup 2 and is deformed after the object has been excreted from the body using consecutively captured images. At this time, the determination unit 5 classifies the state of the object on the basis of a degree of deformation with time of the object. The object changes in nature and state according to a water content thereof. Accordingly, it is possible to classify a state of an object by monitoring a change of the degree of deformation with time of the object.

The determination unit 5 may classify a symptom of a body by comparing a color of an object with a reference on the basis of captured images. The determination unit 5 extracts a feature of a color included in the object on the basis of an analysis result of pixels of the captured images. The determination unit 5 extracts, for example, a change in color of an object as a whole and blood, undigested objects, and the like included in the object. The determination unit 5 determines a color of blood, for example, when blood is extracted. The blood changes in oxidation rate and changes in color with the elapse of time.

The determination unit 5 compares a color of blood extracted from the captured images with a reference of the color of blood and determines a degree of elapse of time of the blood. When it is determined that the degree of elapse of time of the blood is low, the determination unit 5 determines that a bleeding part is present downstream in a digestive organ and extracts a symptom of a body in which blood is discharged downstream in the digestive organ. When it is determined that the degree of elapse of time of the blood is high, the determination unit 5 determines that a bleeding part is present upstream in the digestive organ and extracts a symptom of a body in which blood is discharged upstream in the digestive organ.

The determination unit 5 may extract undigested objects included in the object on the basis of the color of the object. When undigested objects included in the object are extracted, the determination unit 5 extracts a symptom of a body including the undigested objects. Through the aforementioned processing, the determination unit 5 can classify the symptom of the body by comparing the color of the object with a reference. The determination unit 5 may determine a coping method corresponding to the symptom at the same time as classifying the symptom.

The determination unit 5 may classify a symptom of a body by comparing the number of appearances in a predetermined period of the object with a reference. For example, when the number of appearances of the object is less than the reference, the determination unit 5 can extract symptoms associated with constipation or defects of the digestive organ.

A determination method of classifying a state of an object will be described below.

FIG. 11 is a flowchart illustrating a routine which is performed in the determination method of classifying a state of an object. An object in different states is imaged, and training data is prepared on the basis of captured images (Step S10). The training data is subjected to processes such as rotation, reversal, trimming, and zooming and is subjected to data extension of increasing the number of pieces of original data. The determination unit 5 performs iterative learning of classifying the object through deep learning using a convolutional neural network on the basis of the captured images of the object (Step S12).

In the cup 2 of the automatic excretion-processing device 1, the acquisition unit 4 images the object using a camera (Step S14). The acquisition unit 4 acquires captured images of the object and stores the captured images in the storage unit 6 (Step S16). The determination unit 5 extracts a feature of the object and classifies a state of the object on the basis of the acquired captured images of the object with reference to a result of iterative learning using the captured images of the object in different states (Step S18).

As described above, with the automatic excretion-processing device 1, it is possible to automatically classify the state of the object excreted by a care-requiring person on the basis of the captured images obtained by imaging the inside of the cup 2 attached to a body of the care-requiring person. With the automatic excretion-processing device 1, when a space in the cup 2 is small and only a part of the object is captured in the captured images, it is possible to accurately classify the state of the object by determining a change with time of the object based on a determination method using machine learning. With the automatic excretion-processing device 1, it is possible to classify a symptom of the body of the care-requiring person by comparing the state of the object with a reference and to easily manage health of the care-requiring person.

### Second Embodiment

In the first embodiment, the determination unit 5 performs processing in the automatic excretion-processing device 1. A management system 100 in which the automatic excretion-processing device 1 is connected to a network NW and a management device 20 comprehensively manage a care-requiring person may be constructed. In the following description, the same elements as in the first embodiment will be referred to by the same names and reference signs and repeated description thereof will be appropriately omitted.

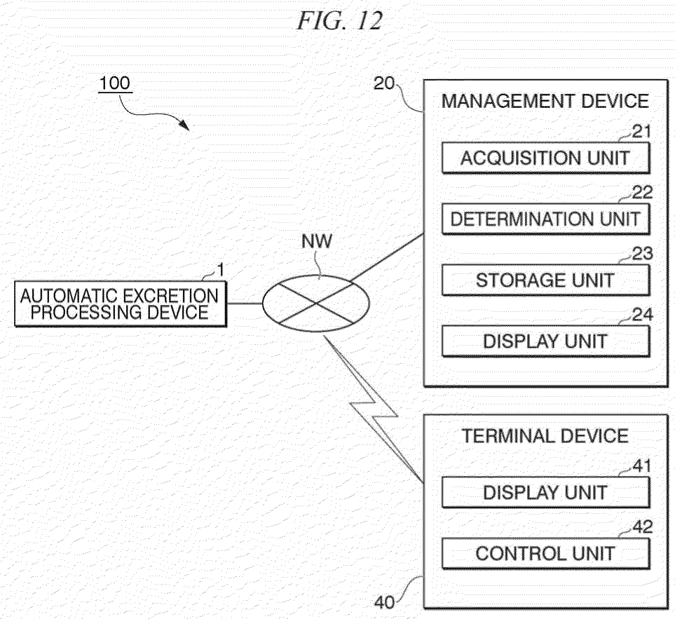

As illustrated in FIG. 12, the management system 100 includes one or more automatic excretion-processing devices 1 connected to the network NW, a management device 20 connected to the network NW, and a terminal device 40 connected to the network NW.

The management device 20 includes, for example, an acquisition unit 21 that acquires a captured image via the network NW, a determination unit 22 that determines characteristics, a state, and the like of the object on the basis of the captured image, a storage unit 23 that stores various types of information, and a display unit 24 that displays a result of determination from the determination unit 22. The management device 20 manages, for example, a plurality of automatic excretion-processing devices 1 which are provided for each building such as a care facility. The management device 20 may manage the plurality of automatic excretion-processing devices 1 for every building.

The determination unit 22 determines and classifies a state of an object on the basis of captured images acquired via the network NW. The captured images are stored as training data in the storage unit 23 after the plurality of automatic excretion-processing devices 1 have started their operations. For example, learning is performed using captured images of objects which have been classified and prepared in advance as training data. The determination unit 5 may start learning without training data, repeatedly perform learning on the basis of captured images acquired from the plurality of automatic excretion-processing devices 1 connected to the network NW, thereby improving determination accuracy with the elapse of time in an operation period.

The determination result from the determination unit 22 may be displayed on a terminal device 40 which is separate from the management device 20. The terminal device 40 is used, for example, by a person engaged in care. The person engaged in care ascertains a symptom or a treatment method of a care-requiring person displayed on the terminal device 40 and performs treatment suitable for the care-requiring person. The terminal device 40 includes a display unit 41 on which a determination result is displayed and a control unit 42 that controls the display unit 41. The terminal device 40 is, for example, a portable information terminal device such as a tablet terminal or a smartphone.

With the management system 100, by collecting the captured images transmitted from one or more automatic excretion-processing devices 1 on the management device 20, the determination unit 22 of the management device 20 performs iterative learning on the basis of the collected captured images, thereby improving determination accuracy. The determination result from the determination unit 22 is comprehensively managed by the management device 20, and thus it is possible to comprehensively manage health states of care-requiring persons using the automatic excretion-processing devices 1. Data associated with the health states of the care-requiring persons is transmitted to the terminal device 40, and a person engaged in care can perform appropriate treatment on the care-requiring persons. With the management system 100, it is possible to automatically manage health states of care-requiring persons and to greatly reduce a burden on a person engaged in care.

While embodiments of the present invention have been described above, the present invention is not limited to the embodiments and can be subjected to various modifications and substitutions without departing from the gist of the present invention. For example, the configurations described in the embodiments and the examples may be combined. In addition to learning using training data according to the aforementioned embodiments, the determination unit 5 may start learning in a state in which there is no training data and repeatedly perform learning on the basis of captured images acquired from the acquisition unit 4, thereby improving determination accuracy.

All or some of the functions of the units in the automatic excretion-processing device 1 and the management system 100 according to the aforementioned embodiments may be realized by recording a program for realizing the functions on a computer-readable recording medium and causing a computer system to read and execute the program recorded on the recording medium. The “computer system” mentioned herein may include an operating system (OS) or hardware such as peripherals.

The “computer-readable recording medium” may be a portable medium such as a flexible disk, a magneto-optical disc, a ROM, a CD-ROM or a storage device such as a hard disk incorporated in a computer system. The “computer-readable recording medium” may include a medium that dynamically holds a program for a short time such as a communication line in a case in which a program is transmitted via a network such as the Internet or a communication circuit line such as a telephone line or a medium that holds a program for a predetermined time such as a volatile memory in a computer system serving as a server or a client in that case. The program may be a program for realizing some of the aforementioned functions or may be a program which can realize the aforementioned functions in combination with another program stored in advance in the computer system.

### REFERENCE SIGNS LIST

| | |
|---|---|
| 1 | Automatic excretion-processing device |
| 2 | Cup |
| 2 | Processing unit |
| 4 | Acquisition unit |
| 5 | Determination unit |
| 6 | Storage unit |
| 7 | Display unit |
| 20 | Management device |
| 21 | Acquisition unit |
| 22 | Determination unit |
| 23 | Storage unit |
| 24 | Display unit |
| 40 | Terminal device |
| 41 | Display unit |
| 42 | Control unit |

1. An automatic excretion-processing device, comprising:
a cup attached to a human body and configured to receive an object which is excreted from the human body;
a processing unit configured to transfer the object in the cup outside of the cup;
an acquisition unit configured to image the inside of the cup and to acquire a captured image of the object; and
a determination unit configured to extract a feature of the object with reference to a result of iterative learning using the captured images of the object in different states on the basis of the captured images acquired by the acquisition unit and to classify the state of the object.

2. The automatic excretion-processing device according to claim 1, wherein the determination unit is configured to monitor a temporal change of the object in the cup on the basis of the captured images and to classify the state of the object.

3. The automatic excretion-processing device according to claim 1, wherein the determination unit is configured to classify the state of the object on the basis of a degree of deformation of the object when the object reaches the inside of the cup and is being deformed on the basis of the captured images.

4. The automatic excretion-processing device according to claim 1, wherein the determination unit is configured to classify a symptom of the human body by comparing a color of the object with a reference on the basis of the captured images.

5. The automatic excretion-processing device according to claim 1, wherein the determination unit is configured to classify a symptom of the human body by comparing a color of the object with a reference on the basis of the captured images and to determine a coping method corresponding to the symptom.

6. The automatic excretion-processing device according to claim 1, wherein the determination unit is configured to extract the feature of the object through the iterative learning in which deep learning using a convolutional neural network is performed and to classify the state of the object.

7. The automatic excretion-processing device according to claim 1, wherein the determination unit is configured to classify a symptom of the human body by comparing the number of appearances of the object in a predetermined period with a reference.

8. A management system comprising:
an automatic excretion-processing device including a cup attached to a human body and configured to receive an object which is excreted from the human body, a processing unit configured to transfer the object in the cup outside of the cup, and an acquisition unit configured to image the inside of the cup and to acquire a captured image of the object; and
a management device configured to acquire a captured image from the automatic excretion-processing device via a network, to extract a feature of the object with reference to a result of iterative learning using the captured images of the object in different states on the basis of the captured images acquired by the acquisition unit and to classify the state of the object.

9. A determination method that is performed by a computer, the determination method comprising:
imaging the inside of a cup in an automatic excretion-processing device including a cup attached to a human body and configured to receive an object which is excreted from the human body and a processing unit configured to transfer the object in the cup outside of the cup;
acquiring a captured image of the object; and
extracting a feature of the object with reference to a result of iterative learning using the captured images of the object in different states on the basis of the acquired captured images and classifying the state of the object.

10. A program causing a computer to perform:
imaging the inside of a cup in an automatic excretion-processing device including a cup attached to a human body and configured to receive an object which is excreted from the human body and a processing unit configured to transfer the object in the cup outside of the cup;
acquiring a captured image of the object; and
extracting a feature of the object with reference to a result of iterative learning using the captured images of the object in different states on the basis of the acquired captured images and classifying the state of the object.
